(19) United States
(12) Patent Application Publication
(10) Pub. No.: US 2009/0008238 A1
(43) Pub. Date: Jan. 8, 2009

(54) TRANSPORTABLE GAS STERILIZATION UNIT, DISPOSABLE GAS GENERATOR, LIGHT ACTIVATED ANTI-INFECTIVE COATING AND METHOD OF DISINFECTION AND STERILIZATION USING CHLORINE DIOXIDE

(75) Inventor: Stuart K. Williams, Tuscon, AZ (US)

Correspondence Address: OBLON, SPIVAK, MCCLELLAND MAIER & NEUSTADT, P.C., 1940 DUKE STREET, ALEXANDRIA, VA, 22314, US

(73) Assignee: THE ARIZONA BD OF REG ON BEHALF OF THE UNIV OF AZ, Tucson, AZ (US)

(21) Appl. No.: 11/547,232

(22) PCT Filed: Apr. 11, 2005
(86) PCT No.: PCT/US2005/012172

§ 371 Date: Sep. 24, 2008

Related U.S. Application Data
(60) Provisional application No. 60/560,909, filed on Apr. 9, 2004.
(60) Provisional application No. 60/561,698, filed on Apr. 13, 2004.

(51) Int. Cl. B01J 19/12; G05B 9/00; G05D 99/00

(52) U.S. Cl. 204/157.48; 422/117; 422/105; 422/129; 423/478; 423/477

(57) ABSTRACT

A transportable gas sterilization unit having a chamber, a disposable gas generator utilizing chlorine dioxide as a sterilant, chemical quencher and a detector and a method of using the unit and to generate chlorine dioxide for medical instrument sterilization or disinfection. A two photon, photo-activated chlorine dioxide system and coatings utilizing chlorine dioxide as at least one sterilant material, and methods for coating medical instruments with the photo activated chlorine dioxide system.

### BACKGROUND OF THE INVENTION

1. Field of the Invention

The invention relates to a transportable sterilization unit and a disposable gas generator utilizing chlorine dioxide as at least one sterilant material, and methods of using the unit and generator for medical instrument disinfection and sterilization. The invention further relates to a two photon photo-activated chlorine dioxide system and coatings utilizing chlorine dioxide as at least one sterilant material, and methods for coating medical instruments with the photo activated chlorine dioxide system.

2. Description of the Related Art

Medical infections remain a major cause of morbidity and mortality in the United States with an estimated healthcare cost of over $60 billion annually. Bacteria and viruses present a huge problem in places such as hospitals and assisted living facilities. Infection control remains a significant problem in both the in-patient and out-patient population. A confounding problem is the incidence of anti-biotic resistant strains of bacteria. More people die every year from infections due to cross-contamination than die from cancer.

An example of the impact of one medical device-related infection is illustrative. Currently in the United States over 5 million catheters are used annually by hospitals. It is estimated that catheters are blamed for 90% of bloodstream infections contracted in U.S. hospitals, or more than 200,000 cases a year. Past studies have shown that 10% to 25% of the infected patients die. A blood infection caused by a catheter can keep a patient in intensive care for nearly 7 days, costing on average, $29,000.

Traditional methods of sterilization include steam, radiation and a variety of chemical sterilizing agents including ethylene oxide, peroxide and ozone. While each of these methods may be used for sterilizing medical instruments, each has certain disadvantages. The drawbacks include limitations on the materials that can be sterilized (e.g steam sterilization cannot be used on many plastics), prohibitive costs, and the presence of post-sterilization chemical residuals. In addition to the disadvantages associated with the actual application of these sterilization methods, there are also inflexibilities associated with the portability of the technologies.

Chlorine dioxide has received attention as a sterilant in recent years and is employed for drinking water disinfection, reducing microbial contamination on fresh food, produce and meats, sanitizing food equipment and for wastewater treatment and slime control in cooling tower waters (see for example Benarde, M. A., Israel, B. M., Olivieri, V. P., and Granstrom, M. L., “Efficiency of Chlorine Dioxide as a Bactericide,” Appl. Microbiol., 13: 776-780, 1965; Olivieri, V. P., Hauchman, F. S., Noss, C. I., and Vast, R., “Mode of Action of Chlorine Dioxide on Selected Viruses,” 619-634, 1985, Chem. Environ. Impact Health Eff. Proc. Conf. 5^{th} 1985, 22; Peeters, J. E., Mazas, E. A., Masschelein, W. J., Villacorta Martiez, d. M., and Debacker, E., “Effect of Disinfection of Drinking Water With Ozone or Chlorine Dioxide on Survival of Cryptosporidium Parvum Oocysts,” Appl. Environ. Microbiol., 55: 1519-1522, 1989; and Roller, S. D., “Some Aspects of the Mode of Action of Chlorine Dioxide on Bacteria,” 1978, The Johns Hopkins University, Baltimore, Md., 1978). Chlorine dioxide has a unique ability to break down phenolic compounds and remove phenolic tastes and odors from water. As such, chlorine dioxide is used in the treatment of drinking water, as well as in wastewater, and for the elimination of cyanides, sulfides, aldehydes, and mercaptans. Another favorable feature is the lack of reaction with ammonia and the fact that chlorine dioxide does not form trihalomethanes or chlorophenols.

Chlorine dioxide gas has been used to sterilize medical devices. Studies have shown that even at low concentrations (20 mg/L), chlorine dioxide is an effective sterilant. Additionally, Rosenblatt and Knapp have reported on the importance of relative humidity for microbial inactivation and conclude that 50% or higher is optimal for sterilization (see Rosenblatt, A. A., and Knapp, J. E., “Chlorine Dioxide Gas Sterilization,” 47-50, 1988, HIMA Conference Proceedings).

One of the most recent applications of the antimicrobial properties of chlorine dioxide involved its utilization for the decontamination in the Hart Senate Office Building, after it received an anthrax contaminated letter in 2001. In 2002, the Brentwood postal plant was also decontaminated using chlorine dioxide after two letters containing anthrax spores passed through the facility resulting in the death of two postal workers dues to inhalation of the spores. The ClO₂ used in these building decontaminations was via large, industrial gas generators.

The 1970's signaled the emergence of chlorine dioxide as a viable commercial product and compound. In 1976, the United States Environmental Protection Agency (EPA) discovered that trihalomethanes (“THMs”) are produced in drinking water as a by-product of chlorination. These THMs are considered carcinogenic. The EPA's Division of Drinking Water began a long-term program to discover replacements for chlorine in drinking water. Of the three leading candidates, chlorine dioxide was judged to be the best overall compound on the basis of high antimicrobial activity, ability to remain in solution, and most importantly, the fact that it does not produce chlorinated organics such as THMs.

Chlorine dioxide's bactericidal activity decreases with lowering of temperature (see Ridenour, G. M. and Armbruster, G. H., “Bactericidal Effect of Chlorine Dioxide,” J. Am. Water Works Assoc., 41:550, 1949) but provides greater sporicidal activity than chlorine. The greater sporicidal activity of chlorine dioxide may be explained by greater utilization of oxidation capacity involving a full change of five electrons (see Ridenour G. M., R. S. Igols, and Armbruster, G. H, “Sporicidal Properties of Chlorine Dioxide,” Water Sewage Works 96: 283, 1949). Chlorine dioxide is an effective water disinfectant for achieving the destruction of bacteria and is also a potent virucide (see Kawanda, Hiroshi, Haneda, and Tadayoshi, “Soil Disinfection by Using Aqueous Chlorine Dioxide Solutions,” (JP95-111095). Apr. 13, 1995; Noss, C. I., Hauchman, F. S., and Olivieri, V. P., “Chlorine Dioxide-Reactivity With Proteins,” Water Res. 20: 351-356, 1986; and Scarpino, P. V., Brigano, F. A. O., Cronier S., and Zink, M. L. “Effects of Particulates on Disinfection of Enteroviruses in Water by Chlorine Dioxide”, EPA-600/2-79-054, 1979, Environmental Protection Technology Series) and is potentially effective against waterborne Cryptosporidium oocytes and its effectiveness is not lessened by increases in the pH of water. Chlorine dioxide shows good antiviral activity at pH 10 in less than 15 seconds (see Berman, D. and Hoff, J. C., “Inactivation of Simian, Rotavirus SA11 by Chlorine, Chlorine Dioxide, and Monochloramine,” Appl. Environ. Microbiol. 48: 317-323, 1984). Solutions of chlorine dioxide have been evaluated against Yersinia enterocolitica and Kiebsiella pneumoniae (see Harakeh, M. S., Berg, J. D., Hoff, J. C., and Matin, A., “Susceptibility of Chemostat-Grown Yersinia Enterocolitica and Klebsiella Pneumoniae to Chlorine Dioxide,” Appl. Environ. Microbiol. 49: 69-72, 1985).

In a major step toward the safe and localized generation of chlorine dioxide from films, wax coatings or dry granules, Bernard Technologies, Inc. in the early 1990 's began developing Microsph□re™. This product line includes controlled release solid-state antimicrobial and deodorizing products that form localized Microatmosph□re™ environments.

Chlorine dioxide is a powerful oxidizer, which must be taken into consideration when choosing the product and packaging materials. Since the reactivity is selective, some materials, such as titanium, stainless steel, silicone rubber, ceramics, polyvinyl chloride, and polyethylene are most likely unaffected by exposure to the gas.

### Chlorine Dioxide Chemistry

Chlorine dioxide chemistry is centered on the conversion of sodium chlorite or sodium chlorate into chlorine dioxide without producing free chlorine. This conversion occurs when ions of chlorite, from sodium chlorite; are acidified with various acid groups. There are a number of related compounds with structure and reactivity described in Table 1:

TABLE 1

| Compound | Symbol | Description |
|---|---|---|
| Sodium Chlorite | NaClO₂ | A primary precursor to chlorine dioxide; “converted” |
| (Chlorite ion) | (ClO₂⁻) | to chlorine dioxide. Chlorite ion is the primary by- |
| | | product of the reaction of chlorine dioxide with other compounds. |
| Sodium Chlorate | NaClO₂ | An oxychlorine compound that may be used in liquid |
| (Chlorate ion) | (ClO₂) | processes to generate gaseous chlorine dioxide. The |
| | | ion can also be a minor component of the by-product |
| | | of the reaction of chlorine dioxide in solution. |
| Chlorous Acid | HClO₂ | A weak acid intermediate in the reaction path |
| | | between sodium chlorite and chlorine dioxide. May |
| | | have high antimicrobial activity, especially when |
| | | combined with chlorine dioxide itself. Produced and |
| | | maintained only under certain conditions of pH and |
| | | concentration in aqueous systems. |
| Chlorine Dioxide | ClO₂ | Powerful oxidizer existing as a gas in nature, 40 |
| | | times more soluble in water than in air; therefore, air |
| | | concentrations are extremely low. |

The chlorite ion and chlorine dioxide are chemically very similar and often referred to as the same entity. The chlorite molecule is converted to chlorine dioxide by going through at least one intermediate compound which is then converted to chlorine dioxide. Under various conditions of concentration and pH, the rate of conversion can vary. Once the chlorine dioxide locates and extracts an electron it is reduced back towards the chlorite ion. The molecular structure of chlorine dioxide and that of its precursor compound chlorite is pictured in FIG. 1.

The generation of chlorine dioxide is based upon the chemical reaction of sodium chlorite and sodium persulfate according to the equation (1):

2NaClO₂+Na₂S₂O₈→2ClO₂+2Na₂SO₄ (1)

The mode of activity of chlorine dioxide is not well understood. Chlorine dioxide exists as a free radical in nature. The activity of chlorine dioxide is believed to stern from the source of the electron extracted by the chlorine dioxide component. At least four specific amino acids readily react with chlorine dioxide: two aromatic amino acids, tryptophane and tyrosine, and two sulfur bearing amino acids, cysteine and methionine. The “ring” structures of tryptophane and tyrosine have a rich source of electrons, which can be captured by strong oxidizers such as chlorine dioxide. The sulfur-bearing amino acids are electronegative and also readily give up electrons.

The oxidative attack on these amino acids is significant. The oxidation of amino acids causes structural disruption of the protein chain, or in the case of the sulfur containing amino acids, a disruption of the disulfide bonds linking several protein chains. This process is shown in FIG. 2. Disulfide bonds, responsible for the structural integrity of the polypeptide molecule, are broken allowing for the two chains to separate. Because the polypeptide must be in a precise three-dimensional shape, this separation “denatures” the protein and renders it inactive. This can directly lead to microbial death. Chlorine dioxide also inactivates many of the cell's enzymes.

### Chlorine Dioxide Health Safety

Many evaluations have shown chlorine dioxide compounds to be non-toxic. Toxicology tests include ingestion of chlorine dioxide in drinking water, additions to tissue culture, injections into the blood, seed disinfection, insect egg disinfection, injections under the skin of animals and into the brains of mice, burns administered to over 1500 rats, and injections into the stalks of plants. “Standard” tests include, Ames Mutation; Chinese Hamster, Rabbits Eye, Skin Abrasion, Pharmacodynamics and Teratology.

Metabolically, both chlorine dioxide and chlorite ions are rapidly reduced following ingestion. Radioactive chlorine tests show that most of the tagged chlorine is excreted from the urine in the form of chlorine ions with a small amount of chlorite ions. The no observed effect level (NOEL) from animal ingestion involving chlorine dioxide and chlorite ions, ranges up to 100 ppm. The half-life for the elimination of chlorine dioxide and chlorite ions from the plasma is less than half that of hypochlorite.

In one study, human volunteers drank chlorine dioxide or chlorite ions in solution, up to a concentration of 24 ppm, and showed no adverse effects. Several studies examined the effects on reproductive toxicity or teratology. There is no evidence of fetal malformation or birth defects at chlorine dioxide concentrations up to 100 ppm, in drinking, as well as via the skin route. With prolonged feeding, toxicity is produced mainly in the red blood cell. Rats fed up to 1000 mg/l of chlorine dioxide chronically for 6 months showed no significant hematological changes. After 9 months, however, red blood cell counts, hematocrit and hemoglobin were decreased in all treatment groups. Lack of toxicity on a long-term, but low-level basis is dramatically illustrated by two separate studies where rats, and honeybees, were fed chlorine dioxide in high doses over a two-year period.

There have been several compounds manufactured incorporating chlorine dioxide in a pharmaceutical preparation. Algicide disinfectant, invented in 1978 is used to disinfect cow teats for preventing mastitis. A chlorine dioxide liquid preparation, Cryoclave, manufactured by International Dioxide, may be used as a treatment for disinfecting the skin. Compounds such as Perchloradoxine, manufactured by Chemical Associates, Inc. and Dura Klor by Rio Linda Chemical Co. are used similarly. Oxyfresh is a dental product containing chlorine dioxide for deodorizing the mouth. Chlorine dioxide has also been combined with medicines taken internally to disinfect the medicine itself, rather than the body. Up to 0.1% of chlorine dioxide or 1,000 ppm was dissolved in one such drug, an antacid from Warner Lambert (see Eichman, M. L. and Belsole, S, “Method for Preserving Antacid Compositions,” Warner-Lambert Company, Morris N.J. Oct. 8, 1974). Allergan Corp. has patented a sodium chlorite composition for disinfecting contact lenses (see Dziabo, A. J., Karageozian, H., and Ripley, P. S., Allergen Inc., (4997626), Mar. 5, 1991). Another patent utilizes chlorine dioxide for the treatment of periodontal disease (see Gordon, G., Kieffer, R., and Rosenblatt, D., “The Chemistry of Chlorine Dioxide, Progress in Inorganic Chemistry,” 1972, 50).

### Bactericidal Effectiveness of Different Reagents Including Chlorine Dioxide

Chlorine dioxide is very effective against a broad range and large variety of microbes including HIV, E. coli, and poliovirus. Some examples of microorganisms known to be controlled by chlorine dioxide, are listed in Table 2.

TABLE 2
Antimicrobial effect of Chlorine Dioxide

| | |
|---|---|
| Viruses | V, Poliovirus, Rotavirus, Herpesvirus, |
| | Echovirus |
| Bacteria | E. coli, Salmonella, Staphylococcus |
| Sore formers | Bacillus spp., Clostridium spp. |
| Molds | Chaetomium, Aspergillus |
| Fungi | Botrytis, Alternaria, Colletotrichum |
| Protozoa | Cryptosporidium, Giardia |

Table 3 below illustrates the bactericidal efficacy of chlorine dioxide relative to other commonly used disinfectants (see Takayama, T., “Bactericidal Activities of Chlorine Dioxide,” J. Antibact. Antifung. Agents, 23: 401-406, 1995). The disinfectants used in the study were Chlorine Dioxide (CD), Glutaraldehyde (GA), Phenol (PN), Absolute Ethyl Alcohol (EtOH), Chlorhexidine digluconate (CHG), Benzalkonium chloride (BAC), Providone iodine (PVP-I) and Sodium hypochlorite (SH). The table provides the minimum bactericidal concentrations in ppm for a 2.5 minute exposure for 5 different organisms. The minimum bactericidal concentrations for chlorine dioxide are significantly lower than for any of the other disinfectants shown.

TABLE 3

| | microorganisms | | | | |
|---|---|---|---|---|---|
| | | | | B. subtilis | |
| Reagents | E. coli | S. aureus | MRSA | (spore) | A. niger |
| GA | 100,000 | 100,000 | 100,000 | 100,000 | 100,000 |
| PN | 10,000 | >10,000 | >10,000 | >10,000 | >10,000 |
| EtOH | 500,000 | 500,000 | 500,000 | 500,000 | 500,000 |
| CHG | 100 | 10 | 1,000 | 1,000 | >10,000 |
| BAC | 100 | 10 | 100 | 1,000 | 10,000 |
| PVP-I | 10 | 100 | 100 | >1,000 | 1,000 |
| SH | 10 | 10 | 10 | >1,000 | 1,000 |
| CD | 1 | 1 | 1 | 100 | 10 |

Many alternative technologies have been researched an attempt to develop a method for preventing bacterial colonization, and the subsequent infection, of medical implants. These include binding antibiotics to the implant, attachment of non-specific antimicrobial agents, such as silver, and modification of the surface texture and composition to prevent bacterial adhesion. Bach of these techniques has met with limited success, with complications including bacterial resistance to antibiotics, and maintenance of the bactericidal concentrations of the anti-infective agent.

The generation of chlorine dioxide from conventional starting materials such as sodium chlorite or sodium chlorate and an acid requires that the chlorine-containing material be mixed with the acid. If the chlorine-containing material is not separated from the acid then chlorine dioxide will immediately form. Thus, before using chlorine dioxide in conventional sterilization processes it is necessary to prepare a mixture of a chlorine-containing compound and an acid.

Photoacid generating chemistries have been examined for utilization in the areas of 3D-microfabrication, ultra-high-density optical data storage, biological imaging, and the controlled release of biological agents (see Zhou; W., Kuebler, S. M., Braun, K. L., Yu, T., Cammack, J. K., Ober, C. K., Perry, J. W., Marder, S. R., “An Efficient Two-Photon-Generated Photoacid Applied to Positive-Tone 3D Microfabrication,” Science, 296: 1106-1109, 2002). The generation of acid occurs when the PAG chemistry adsorbs photons from an applied light source, which causes the release of protons. In terms of the generation of chlorine dioxide, these protons cause the oxidation of NaClO₂, and the subsequent production of ClO₂ and Na+ ions.

Light-activated release of chlorine dioxide from hydrogels differs from previously described techniques in many ways. Most significantly, there is the established effectiveness of chlorine dioxide against a broad range of microbes (bacteria, viruses, mold and fungi) and chlorine dioxide's ability to kill resistant strains and antibiotic and other biocide resistant organisms.

### SUMMARY OF THE INVENTION

One embodiment of the invention includes a portable gas sterilization unit for generating chlorine dioxide gas for sterilizing or disinfecting articles such as medical devices. The portable gas sterilization unit includes a chamber having a door and a chlorine dioxide generator that may be operated at ambient temperatures and does not create toxic by-product gases or chemical residuals.

In an embodiment of the invention the portable gas sterilization unit may be used in a method for sterilizing or disinfecting reusable medical instruments by exposing the medical instruments to an atmosphere containing chlorine dioxide gas.

Another embodiment of the invention includes a light-activated chlorine dioxide system that produces chlorine dioxide gas when exposed to light, such as fluorescent lighting.

Another embodiment of the invention includes a light activated chlorine dioxide releasing material. The material may be placed on the surface of medical instruments or devices to perform an anti-infective or sterilant function.

### BRIEF DESCRIPTION OF THE DRAWINGS

FIG. 1 shows the molecular structure of chloride dioxide and a precursor compound thereof;

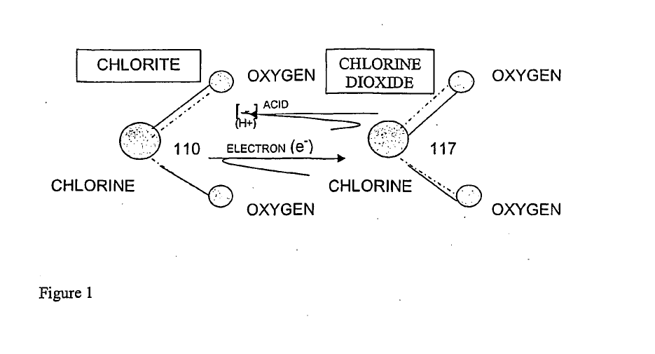

FIG. 2 shows the reaction of chlorine dioxide with a disulfide bond of a protein chain;

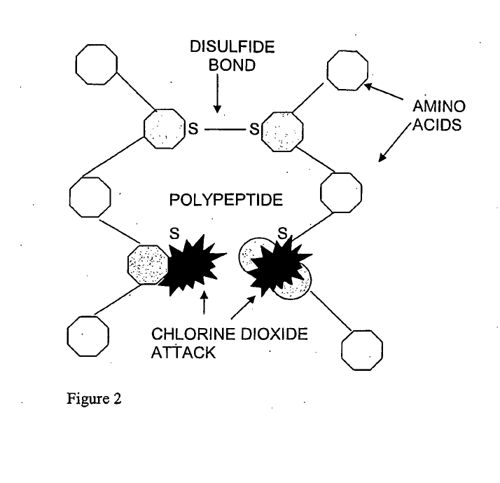

FIG. 3 shows a disposable chlorine dioxide generator unit;

FIG. 4 shows a portable chlorine dioxide gas sterilization unit;

FIG. 5 shows a chemical quencher;

FIG. 6 shows a two component disposable chlorine dioxide generator;

FIG. 7 shows a chlorine dioxide gas generating system in an incubator;

FIG. 8 shows gas generation in a chlorine dioxide gas generator;

FIG. 9 shows a gas sterilization unit;

FIG. 10 shows a partially pressurized sterilization chamber;

FIG. 11 shows a gas sterilization cabinet;

FIG. 12 shows a light activated system for generating chlorine dioxide.

### DETAILED DESCRIPTION OF THE PREFERRED EMBODIMENTS

### Portable Gas Sterilization Unit

In one embodiment the portable gas sterilization unit of the invention includes at least a chamber, a chlorine dioxide detector, a disposable chlorine dioxide generator, and a chemical quencher. In embodiments, the portable gas sterilization unit may contain a plurality of chambers, chlorine dioxide detectors, disposable chlorine dioxide generators, and/or chemical quenchers. The portable gas sterilization unit may be used for sterilization and/or disinfection.

The portable gas sterilization unit is portable or transportable. In embodiments the portable gas sterilization unit may be moved, and readied for operation by one person in a matter of minutes or hours. The portable gas sterilization unit maybe transported by vehicle and may provide sterilization services in a moving vehicle. In one embodiment the portable gas sterilization unit may be collapsed for transport and later readily assembled. The portable gas sterilization unit may be made of components that can be easily assembled under severe conditions.

The chamber comprises a shell and at least one door. The shell may be in the form of any three dimensional shape. Preferred embodiments include a box having one or more flat and/or curved surfaces, a sphere or a spherical form. The shell is made of at least one rigid surface, preferably each surface of the shell is rigid. A rigid surface is a surface that holds its shape under ordinary handling conditions.

The shell is made from a solid rigid material. Examples of solid rigid materials include cardboard, thermoplastics, thermosets, metals, natural materials such as wood and stone, concrete, and any other material which may hold its shape and/or structure under ordinary handling conditions. The solid rigid material is preferably a metal such as steel, stainless steel, aluminum or a mixture of metals. The solid rigid material may be glass which provides an advantage if a transparent shell is desired. The solid rigid material may be a plastic including transparent plastics such as polycarbonate and acrylic, or a semitransparent plastic material such as a polyolefin, for example, polyethylene, polypropylene, polybutene, polyvinylchloride, mixtures thereof, and copolymers thereof. The solid rigid material may also be an opaque thermoplastic or thermoset material such as a cured epoxy or acrylic.

In a different embodiment of the invention the shell comprises a non-rigid material supported by a rigid frame. The non-rigid material may be, for example, a bag or a sheet or other non-rigid covering such as a woven fabric or extruded sheet or film such as mylar which is supported by a frame made of one or more of the solid rigid materials described above.

The shell has at least one opening through which articles may be transferred to an interior chamber of the shell for sterilizing. The opening is large enough so that articles such as medical devices may be passed into the interior of the shell. The opening can be closed and sealed by a door connected to the shell. Closing the door encloses the interior chamber of the shell. The door may be made of the same solid rigid material as the shell or a different solid rigid material. The door is fitted to the shell so that a seal may form between the surfaces of the door which contact the surfaces of the shell, for example, through a gasket, liquid or electromagnetic contact. The shell provides a gas tight seal when the door of the shell is closed and when there are no other open or unobstructed openings from the interior of the shell to the outside of the shell. A gas tight seal is a seal that holds a pressure of up to two psi (lbs./in²) for a period of up to 24 hours with less than a 5% decrease in pressure at standard conditions. Preferably, there is less than 1% change in pressure and more preferably there is no measurable change in pressure over a period of 24 hours at standard conditions.

The door and the shell are fitted with a locking mechanism. The locking mechanism permits the interior of the chamber to be held closed by the door with a gas-tight seal. The locking mechanism is electronically controlled through a feedback loop to the detector (described below). The door is locked so that the door closing the interior chamber of the shell cannot be opened. After the door has been closed and the locking mechanism engaged, the door may only be opened under certain predefined conditions so that chlorine dioxide gas is not accidentally allowed to escape from the chamber interior. The predefined conditions may include the position of the door, the period of time elapsed during a sterilization procedure, the presence of chlorine dioxide gas and/or the condition of the gas quencher (described below). For example, when the detector registers that chlorine dioxide gas is present in the interior chamber of the shell, the locking mechanism may engage the lock so that it can not be opened until the detector determines that no chlorine dioxide present. Until the detector no longer records the presence of chlorine dioxide gas, or when the detector registers that the concentration of chlorine dioxide gas is below a threshold limit, the locking mechanism is disengaged and the door may be opened permitting access to the interior of the chamber.

The portable gas sterilization unit further comprises a chlorine dioxide detector. The chlorine dioxide detector is connected to the interior chamber of the shell. The chlorine dioxide detector may be mounted in the interior of the shell or may be mounted so that the display portion of the detector is mounted on an outside surface of the shell or remotely from the shell. The chlorine dioxide detector is capable of measuring chlorine dioxide concentrations in gaseous environment through a range of concentrations of, for example, 0-3,000 ppm; 10-2,000 ppm; 50-1,000 ppm; 100-500 ppm; and all values between the stated values.

The chlorine dioxide detector may include a UV source and a detector that measures chlorine dioxide concentration by a maximum absorption at approximately 365 nm. The chlorine dioxide detector may also be a detector such as a mass spectrometric detector, chromatographic, ultraviolet, infrared or other detector which is sensitive to any absorption or emission of electromagnetic or radiative energy from chlorine dioxide or the physical presence of chlorine dioxide gas (e.g., mass).

The chlorine dioxide detector of the portable gas sterilization unit may be a commercially available chlorine dioxide detector. For example the detector may be a chlorine detector manufactured by City Technology, Ltd; (England), modified to detect chlorine. The detector is connected to the chamber of the portable gas sterilization unit through a hose or conduit which may allow gases to pass between the chamber interior to the detector. The detector may be connected to an air pump system to permit the flow of gases through the detector. The detector may be mounted inside the chamber or outside the chamber of varying sizes. The detector may be one which directly provides a signal that is converted to ppm ClO₂ or an analog signal that is converted to and data is collected on a computer.

The portable gas sterilization unit includes a disposable chlorine dioxide generator. The disposable chlorine dioxide generator is connected to the interior of the shell through at least one opening. Preferably, the disposable chlorine dioxide generator is connected to the interior of the shell through two connections which pass through both inner and outer surfaces of the shell through orifices present in the shell. The disposable chlorine dioxide generator may comprise a chemical chamber that is separate from but connected to the shell. The chemical chamber may hold and/or mix one or more reactants capable of generating chlorine dioxide gas. The disposable chlorine dioxide generator may include those which are mounted to the chlorine dioxide detector system on a reaction jar.

The reactants capable of generating chlorine dioxide gas include at least one of sodium chlorite and sodium chlorate, and an acid. The reactants may be present in solid form, liquid form, or a combination thereof. In a preferred embodiment one or more of the materials is present as a first compound in an ampoule placed inside a sealed flexible tube. A second compound is present in the sealed tube but separated from the first compound in the ampoule. Upon breaking the ampoule inside the flexible tube the second compound is permitted to mix with the first compound in the ampoule thereby permitting mixing of the first and second compounds and causing a chemical reaction. For example, the ampoule may contain at least one of sodium chlorite or sodium chlorate and the flexible sealed plastic tube may contain an acid such as an aqueous hydrochloric acid solution upon mixing of the sodium chlorite or sodium chlorate with the aqueous acid solution. Chlorine dioxide is generated. The chlorine dioxide may be released from the sealed plastic tube by puncturing the tube with a needle or other device present inside the chemical chamber. The plastic tube may be capped at one end with a membrane permeable to ClO₂ to permit the release of the chlorine dioxide.

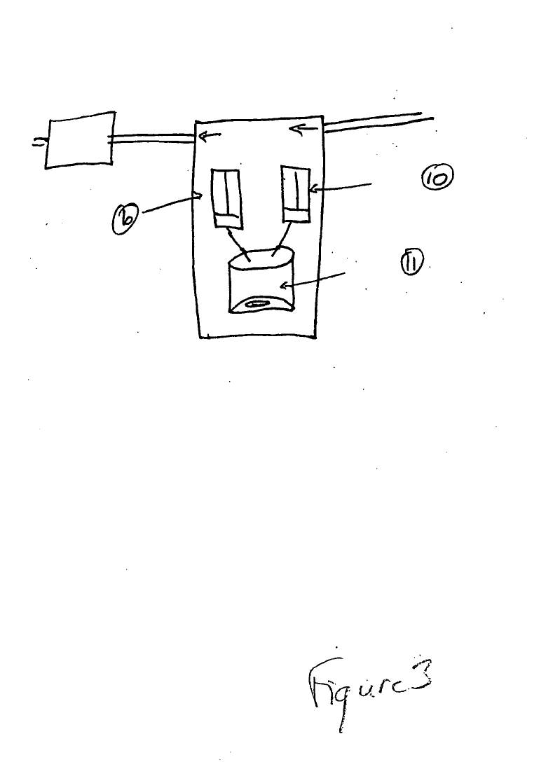

In another embodiment of the invention the disposable chlorine dioxide generator mixes two liquids in a reaction chamber to form chlorine dioxide gas. The two liquids are stored separately, for example in separate syringes, to permit their accurate metering into the reaction chamber. The reaction chamber may include an impeller or magnetic stirring device to ensure good mixing of the liquid solutions dispensed into the reaction chamber. For example, a first reaction liquid may contain a solution containing one or more of dissolved sodium chlorite or sodium chlorate. A second reaction liquid may contain an acid such as an organic or inorganic acid in pure form or diluted with an aqueous or organic diluent. The reaction chamber may be connected to two passages which are connected to the interior chamber of the shell. A pump, fan or other device for moving the gaseous materials formed from the reaction of the liquids in the reaction chamber permits circulation of the gases evolved in the reaction chamber through the interior chamber of the shell. Thus the chlorine dioxide generated by mixing the first and second liquids in the chemical reaction chamber may be transferred into the interior of the shell without escape of chlorine dioxide outside the portable gas sterilization unit. The disposable gas generator is shown in FIG. 3 reference no. 10 is a syringe or container for holding first and second reactants. The reactants are mixed in a mixing chamber shown as reference no. 3.

The portable gas sterilization unit permits the production of controlled amounts of chlorine dioxide in a sterilization chamber (e.g., the interior of the shell) without allowing escape of chlorine dioxide into the atmosphere surrounding the portable gas sterilization unit. The connections between the chemical reaction chamber and the interior of the shell may be fitted with one or more valves to permit or block passage of the reaction chamber gases into the interior of the shell. In a preferred embodiment the chemical reaction chamber is connected to the interior of the shell through two orifices. The orifices may be connected to the chemical reaction chamber by two different tubes. The tubes are then connected to the chemical reaction chamber at points which permit gas exchange through the chemical reaction chamber. The chlorine dioxide-containing gases may be circulated from the reaction chamber of the disposable gas generator through the interior of the chamber of the shell. The atmosphere from the interior of the shell is forced through the chemical reaction chamber of the disposable gas generator in one direction through a loop defined by the chemical reaction chamber, through a first tube into the interior of the shell, through a first orifice, then through the interior of the shell and then to exit the interior of the shell at a second orifice connected to a second tube which enters the chemical reaction chamber of the disposable gas generator.

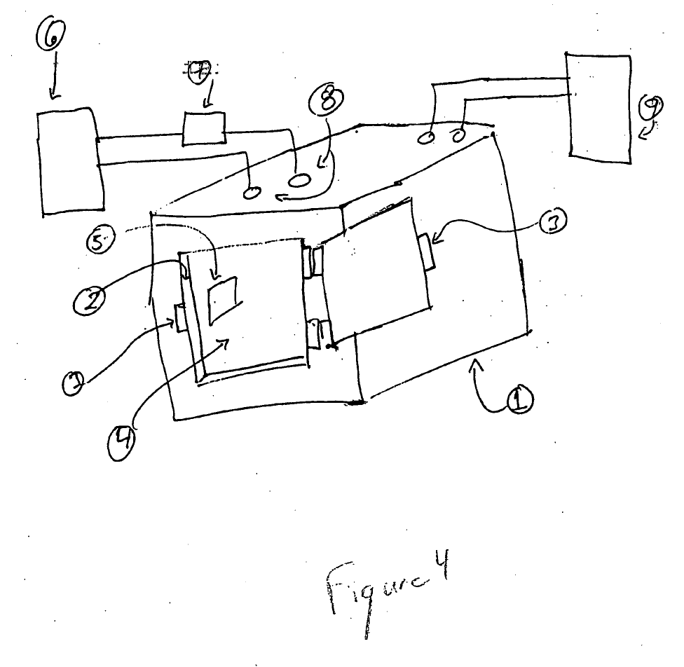

The portable gas sterilization unit is shown in one embodiment in FIG. 4 reference no. 1 identifies the shell made from the solid rigid material. In the embodiment shown in FIG. 4 the shell is in the form of a box similar in size and dimensions to conventional autoclaves used for heat and/or steam sterilization. The solid rigid material making up the walls of the portable gas sterilization unit are shown as reference numeral 2. The blocking mechanism is shown as 3 and contains reference numeral 3a affixed to the shell of the portable gas sterilization unit and reference number 3b affixed to the door. The interior of the chamber is surrounded by the rigid shell and is shown as reference numeral 4. The chlorine dioxide detector is mounted in the embodiment shown in FIG. 4 in the interior chamber of the shell. A disposable gas generation unit is shown as 6 and is connected to the shell by two passages. One of the passages passes through a fan, impeller or device for moving gases through the interior chamber of the shell through the reaction chamber of the disposable gas generator. The orifices through which the chlorine dioxide generated in the disposable chlorine dioxide generator pass into an out and of the interior chamber of the portable gas sterilization unit are shown as 8. The quencher is shown as 9 and is connected to the shell of the portable gas sterilization unit through two orifices penetrating through the solid rigid material of the shell.

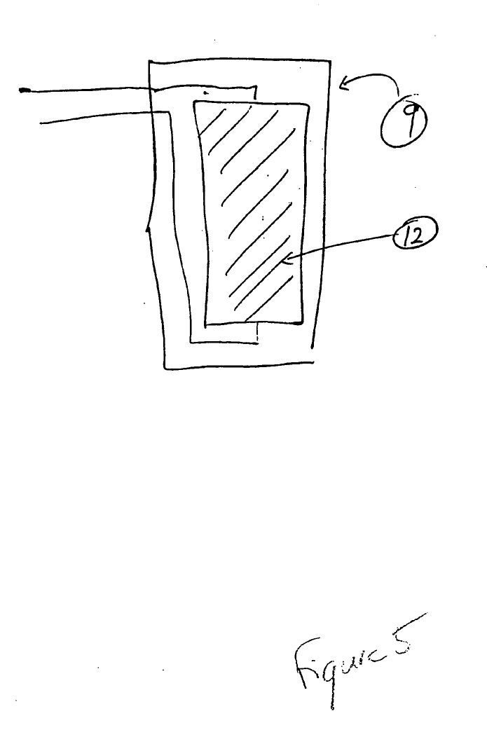

The quencher is shown as FIG. 5. Passages identified as 13 carry gases from the interior chamber of the portable gas sterilization through the quencher. A fan, impeller or other means of moving gases through the quencher may be present in the passage between the quencher and the shell. A chemical material may be present, for example, in the form of a cartridge. 12 inside the quencher.

The disposable chlorine dioxide generator may be one that permits a single sterilization process to be carried out or multiple sterilization/disinfection processes to be carried out with or without recharging the reactants. In a preferred embodiment all of the reactants present in the chemical reaction chamber are completely reacted produce only non-toxic end products such as acidic salt solutions.

During the reaction to form chlorine dioxide it is preferable that the acid material is added in excess to any sodium chlorite and/or sodium chlorate to ensure that complete chlorine dioxide evolution has occurred and that no unreacted sodium chlorite and/or sodium chlorate is present in the reaction chamber after a sterilization has been completed. In a preferred embodiment the entire chemical reaction chamber is a disposable cartridge that permits simple disposal of the by-products of chlorine dioxide generation. The disposable cartridge allows liquids to be dispensed therein and mixed to generate chlorine dioxide. The cartridge is removed subsequent to reaction and subsequent to the sterilization procedure. After the chlorine dioxide generation is complete, the cartridge may be discarded safely because no potential for chlorine dioxide generation remains.

In another embodiment of the invention the disposable chlorine dioxide generator consists of an ampoule type chlorine dioxide generation system. Such ampoule systems are disclosed in published U.S. Application No. 2004/021065, incorporated herein by reference in its entirety.

The portable gas sterilization unit further comprises a chemical quencher. The chemical quencher is connected to the interior of the chamber through one or more orifices. In embodiments the chemical quencher is attached to the portable gas sterilization unit through the tubes or connections between the shell and the disposable gas generator. The chemical quencher may connect to the shell through an orifice that permits gas flow from the interior of the chamber to the atmosphere (e.g., a vent). The atmosphere inside the portable gas sterilization unit may be moved through the chemical quenching system to remove any residual chlorine dioxide gas. The chemical quenching system may be in the form of, for example, a cartridge loaded with an absorbing, adsorbing, chemically reactive material or a combination thereof. Examples of the material that may be present in the chemical quencher include materials such as iron, iron oxide, carbon black, caustic water, and oil.

As the chlorine dioxide exits the interior chamber of the shell through the chemical quencher, residual chlorine dioxide gas and is captured, absorbed, adsorbed or reacted by the material in the chemical quencher. Preferably, the chemical quencher removes all of the chlorine dioxide from the atmosphere present in the interior of the chamber of the portable gas sterilization unit and permits the escape of only inert components of the ambient atmosphere. The chemical quencher may reduce the amount of chlorine dioxide by 98%, preferably 99%, even more preferably 99.5% based upon the total amount of chlorine dioxide remaining in the interior of the portable gas sterilization unit after a sterilization run. Most preferably the chemical quencher removes all of the chlorine dioxide gas remaining in the interior of the chamber after a sterilization has been completed.

The portable gas sterilization unit may be used to sterilize, for example, medical instruments. A medical instrument or device, such as a suture, may be placed in the interior of chamber of the shell when the door is open. The door is subsequently closed and a sterilization run is initiated electronically. The locking mechanism locks the door thereby sealing the atmosphere of the interior of the shell. Chlorine dioxide is then generated by the chlorine dioxide generator. The chlorine dioxide gas is circulated through the interior of the shell in an amount to disinfect or sterilize the medical article or device. After reaching a threshold maximum chlorine dioxide concentration as measured by the detector or as limited by the maximum theoretical amount of chlorine dioxide that may be formed by the disposable chlorine dioxide generator; the gases present in the interior of the shell are passed through the chemical quenching system and exhausted after residual chlorine dioxide has been removed.

### Photoactivated Chlorine Dioxide System

Another embodiment of the invention includes a light activated chlorine dioxide system (e.g., photo activated chlorine dioxide system). The light activated chlorine dioxide system includes a chlorine component and an acid component. The chlorine component may include one or more of sodium chlorate and sodium chlorite. The chlorine component may be present in a pure form or present as a mixture or solution with an inert diluent or a co-reactant. For example, sodium chlorate and/or sodium chlorite may be present as a solution in water. Sodium chlorate and/or sodium chlorite may also be present together or individually as solid materials.

The light activated chlorine dioxide system also has a photo acid component. The photo acid component is a two-photon photo acid component. Any two-photon photo acids may be used as the two-photon photo acid component of the invention. The two-photon photo acid components described in U.S. Published Application No. 2003/0235605 (incorporated herein by reference in its entirety) may be used individually or in combinations.

A preferred two photon photo acid component is diphenyliodonium 9,10-dimethoxyanthracenesulfonate (structural formula shown below), which is commercially available from Sigma-Aldrich.

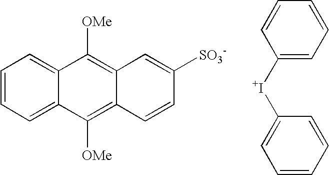

In one embodiment the chlorine component and photo acid component are present as a mixture with one another. The mixture may be a mixture of solid materials or a homogenous solution of the chlorine and photo acid component. In this embodiment of the invention the mixture of materials is shielded from light until the generation of chlorine dioxide is desired. Upon exposure of the mixture of the chlorine component and photo acid component to light the photo acid component produces an acid. The acid reacts with the sodium chlorite and/or sodium chloride to form chlorine dioxide.

In another embodiment of the invention the chlorine component and the photo acid component are present in separate containers are or otherwise separated so that intimate contact between the chlorine component and the photo acid component is not possible. In a preferred embodiment two solutions, one each of the chlorine component and the other of the photo acid component, are kept separate and mixed when needed. After mixing the resulting mixture is exposed to light and thereafter releases chlorine dioxide gas. The chlorine dioxide gas formed by exposure to light may be released directly from the mixture or may be captured and/or dissolved in a matrix material within which the chlorine component and the photo acid component are dispersed.

The chlorine component and the photo acid component may be dispersed in, for example, a hydrogel. Depending upon the viscosity of the material making up the hydrogel the chlorine dioxide gas may escape directly into the surrounding atmosphere or may alternatively be captured and transiently trapped in the hydrogel (e.g., a semifluid viscous matrix). Chlorine dioxide can then escape slowly in measured amounts from the matrix material into the atmosphere or environment surrounding the hydrogel. In this form the photoactivated chlorine dioxide system may be used as a salve or ointment on, for example, wounds for disinfection or sterilization.

The chlorine and photo acid components may be also be present as mixtures dispersed in, for example, a coating matrix. For example, the chlorine and photo acid components may be co-extruded with a matrix such as a thermoplastic resin or other material that becomes solid at room temperature. The resulting mixture may be used to coat a surface. When subsequently exposed to light the coated surface releases chlorine dioxide gas which functions to disinfect or sterilize the article or substrate having the coated surface.

An embodiment of the invention includes coating a medical device or instrument with a chlorine dioxide-generating coating comprising the chlorine and photo acid components dispersed therein. The coated article or medical device is stored in a light-fast covering until needed. When the article is removed from the container and exposed to light, it self-sterilizes or self-disinfects.

### EXAMPLES

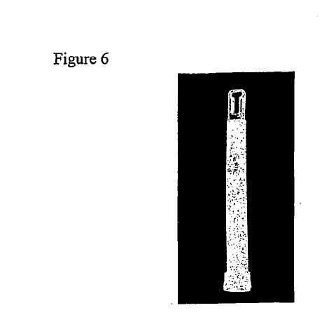

A two component disposable chlorine dioxide generator is illustrated in FIG. 6. The generator used NaClO₂ as the major reactant. The NaClO₂ was present as a 30% aqueous solution (w/v) and placed in a thin-walled glass tube (e.g., ampoule) sealed at each end. The volume of the glass tube was about 1.5 ml. The glass ampoule was placed in a flexible plastic tube containing tartaric acid powder in excess. Upon breaking the glass ampoule by bending the plastic outer tube the NaClO₂ mixed with tartaric acid releasing ClO₂ gas.

The concentration of chlorine dioxide generated is determined by altering the concentration of sodium chlorite solution, flow rate and air flow rate, to permit chlorine dioxide generation using the following equation:

$$Cg = 1.9812 \times 103 \frac{{C}_{s} {F}_{s}}{{F}_{g}}$$

where:

Cg is the theoretical output concentration of chlorine dioxide (ppm)

Cs is the concentration of sodium chlorite solution (%)

Fs is the sodium chlorite solution flow rate (ml/min)

Fg is the total airflow rate (l/min)

Using titration procedure of the chlorine dioxide output, the aforementioned parameters can be adjusted to obtain the desired chlorine dioxide concentration. Chlorine dioxide concentrations in the range of 1 to 2000 ppm have been achieved at a constant concentration of ClO2 in excess of 24 hours.

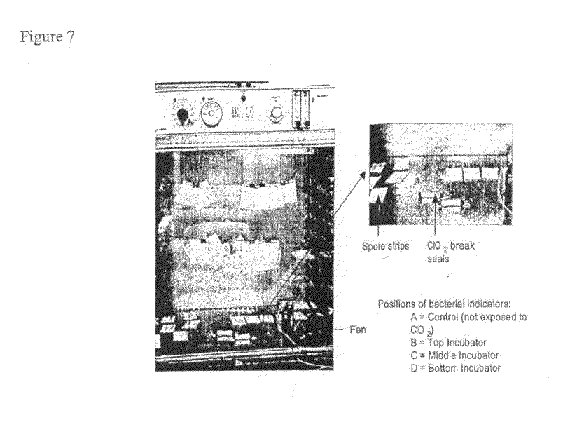

In the first series of evaluations the gas generating system was placed in a 37° C. incubator, as illustrated in FIG. 7. This experimental system was based on a tissue culture incubator that was not gas tight.

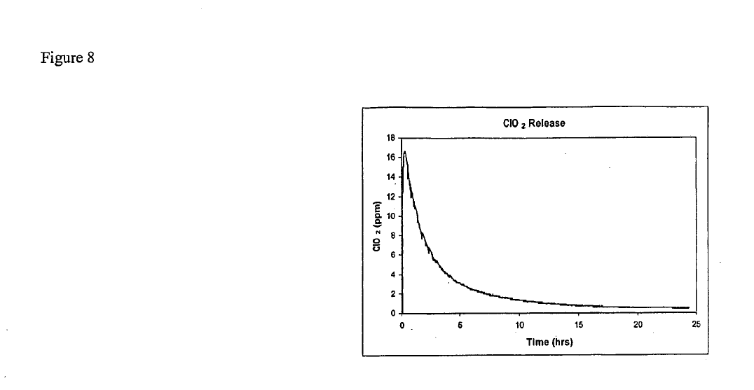

The gas generation by these disposable device prototypes was very rapid as illustrated by FIG. 8.

During these experiments test strips containing either B. subtilis (106 spores/strip) and B. stearothermophilus (log values between 103 and 107 spores/strip) were placed in the incubator. Following a 24 hour exposure to ClO₂ generated by the disposable chlorine dioxide generator, test strips were incubated in appropriate media and cell growth analyzed. The experimental results indicate that the concentrations of chlorine dioxide gas generated resulted in significant sporicidal activity, as is illustrated by the data in Table 4 and Table 5.

TABLE 4
B. subtilus sporicidal activity

| Experimental | | | | | | | | |
|---|---|---|---|---|---|---|---|---|
| Conditions | | Day 1 | Day 2 | Day 3 | Day 4 | Day 5 | Day 6 | Day 7 |
| A | 1 | +/− | + | + | + | + | + | + |
| | 2 | +/− | + | + | + | + | + | + |
| | 3 | +/− | + | + | + | + | + | + |
| | 4 | +/− | + | + | + | + | + | + |
| | 5 | +/− | + | + | + | + | + | + |
| B | 1 | − | +/− | + | + | + | + | + |
| | 2 | − | − | − | − | − | − | − |
| | 3 | − | − | − | − | − | − | − |
| | 4 | − | − | − | − | − | − | − |
| | 5 | − | − | − | − | − | − | − |
| C | 1 | − | − | − | − | − | − | − |
| | 2 | − | − | − | − | − | − | − |
| | 3 | − | +/− | + | + | + | + | + |
| | 4 | − | − | − | − | − | − | − |
| | 5 | − | − | − | − | − | − | − |
| D | 1 | − | − | − | − | − | − | − |
| | 2 | − | − | − | − | − | − | − |
| | 3 | − | − | − | − | − | − | − |
| | 4 | − | − | − | − | − | − | − |
| | 5 | − | − | − | − | − | − | − |

| | | | | | | | |
|---|---|---|---|---|---|---|---|
| Positive | + | + | + | + | + | + | + |
| Control | | | | | | | |
| Media Sterility | − | − | − | − | − | − | − |
| Control | | | | | | | |

TABLE 5
B. stearothermophilus sporicidal activity

| Experimental | | | | | | | | |
|---|---|---|---|---|---|---|---|---|
| Conditions | | Day 1 | Day 2 | Day 3 | Day 4 | Day 5 | Day 6 | Day 7 |
| A | 10³ | +/− | + | + | + | + | + | + |
| | 10⁴ | +/− | + | + | + | + | + | + |
| | 10⁵ | +/− | + | + | + | + | + | + |
| | 10⁶ | +/− | + | + | + | + | + | + |
| | 10⁷ | +/− | + | + | + | + | + | + |
| B | 10³ | − | − | − | − | − | − | − |
| | 10⁴ | − | − | − | − | − | − | − |
| | 10⁵ | − | − | − | − | − | − | − |
| | 10⁶ | − | − | − | − | − | − | − |
| | 10⁷ | − | − | − | − | − | − | − |
| C | 10³ | − | − | − | − | − | − | − |
| | 10⁴ | − | − | − | − | − | − | − |
| | 10⁵ | − | − | − | − | − | − | − |
| | 10⁶ | − | − | − | − | − | − | − |
| | 10⁷ | − | − | − | − | − | − | − |
| D | 10³ | − | − | − | − | − | − | − |
| | 10⁴ | − | − | − | − | − | − | − |
| | 10⁵ | − | − | − | − | − | − | − |
| | 10⁶ | − | − | − | − | − | − | − |
| | 10⁷ | − | − | − | − | − | − | − |

| | | | | | | | |
|---|---|---|---|---|---|---|---|
| Positive | + | + | + | + | + | + | + |
| Control | | | | | | | |
| Media Sterility | − | − | − | − | − | − | − |
| Control | | | | | | | |

Transportable Sterilization System

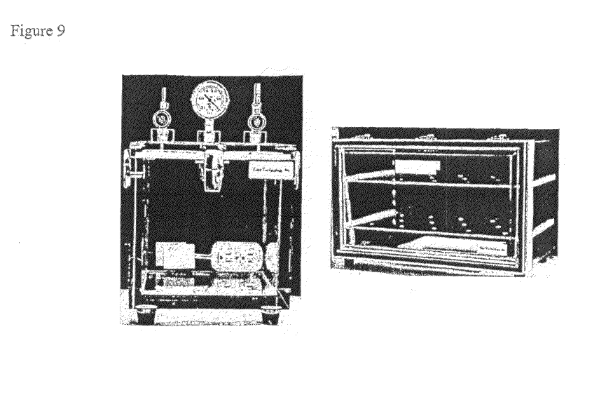

Two chlorine dioxide gas sterilization units were assembled and modified to permit assessment of ClO₂ efficacy. These two prototypes are illustrated in FIG. 9. The first prototype permits mounting of multiple sensors and measurement devices. This cabinet can also be maintained under a significant positive pressure. The second cabinet is a final sterilization system. The gas generation system was not mounted on this device.

To determine the operating parameters for the chlorine dioxide sterilization unit, a series of evaluations are necessary to assess the concentration and exposure times of chlorine dioxide required to kill B. subtilis. This is an EPA standard organism used to evaluate sterilization systems. The first experiment determines the optimal concentration and time parameters using a permanent gas generation system. The second series of studies evaluates a disposable gas generation system constructed to produce effective gas concentrations identified in experiment 1.

### Experiment 1

### Design & Construction of a Chlorine Dioxide Gas Sterilization Unit (PSU)

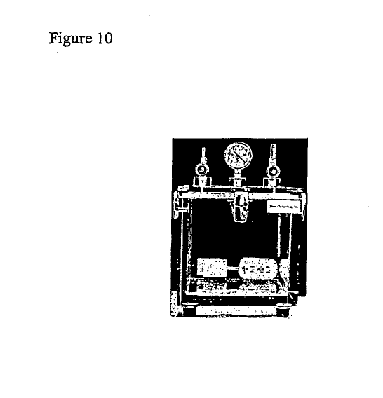

A commercially available plexiglass chamber was modified to accept numerous access ports. This chamber is gas tight and can be partially pressurized (FIG. 10). The chamber was modified to pen-nit continuous flow of chlorine dioxide throughout the chamber. Additional ports may be mounted to permit constant assessment of ClO₂ concentration using a modified ClO₂ sensor, and to permit evaluation of chamber humidity, temperature and pressure.

Prior to exposure to ClO₂ the chamber was loaded with B. subtilis spore strips with 106 spores/strip (Ravenlabs, Inc.), sealed in Tyvek bags. All experiments were conducted at room temperature and atmospheric pressure. The concentration of chlorine dioxide levels generated and the exposure times were varied for each experiment. Each pair of the concentration and time values outlined in Table 6 were evaluated in triplicate.

TABLE 6
ClO₂ concentration and time.

| Parameter | Concentration/Duration |
|---|---|
| Chloride Dioxide Concentration | 25, 50, 100, 200, 500, 1000, 1500 |
| (ppm) | |
| Exposure time (hrs) | 0.5, 1, 2, 4, 6, 12, 24 |

Following exposure to ClO₂ the spore strips were removed from the Tyvek bags and placed in a growth indicator solution in Tryptic Soy Broth (RavenLabs, Inc.) and incubated at 37° C. for seven days. Bacterial growth was assessed visually. Conditions that result in cultures that remain bacteria free for seven days are considered effective.

A disposable gas generation system was evaluated for sporicidal effectiveness. This disposable generator is described and illustrated above. The production of ClO₂ by this system is controlled by varying the amount of aqueous ClO₂ placed in the breakable glass ampoule. The first series of studies in this experiment evaluated ClO₂ production by disposable generators assembled using aqueous ClO₂ of varying volumes to establish steady state ClO₂ concentration identified as effective in experiment 1. These generators contained excess tartaric acid and for this reason only the ClO₂ concentration was varied.

### Evaluation of the Effect of ClO₂ Sterilization on Medical Instrument Material Characteristics.

Materials for the manufacture of medical instruments are subjected to sterilization followed by use of analytical methods to determine changes in the materials and in vitro methods to assess material biocompatibility. The analytical and biocompatibility studies provides an initial assessment of the effect of ClO₂ on materials.

### Experiment 3

### Chemical Analysis

Material samples of stainless steel and high density polyethylene, sterilized are analyzed using Auger electron spectroscopy (AES) to determine the effects of the chlorine dioxide sterilization procedure on the surface chemistry of the materials. AES is a surface sensitive analytical method for assessing the elemental composition of the outermost atomic layer of solid materials, including metals and organic substances. AES involves measuring the number of emitted electrons as a function of kinetic energy, in response to applied incident radiation. The energy associated with the emitted electrons is characteristic of the element from which it originates. The principle advantages of AES over other surface analysis methods include excellent spatial resolution (<1 mm) and surface sensitivity (˜20 Å). The depth of penetration into the sample is of the order of 2-3 nm. From these measurements, it is possible to isolate any differences in the surface chemistry of the materials pre- and post-sterilization with chlorine dioxide. Sample sizes are in the order of 1.5 cm in diameter and 0.5 cm high. There is no requirement for material preparation prior to AES analysis, and the procedure usually takes under 5 minutes for a complete-survey spectrum from 0-2000 eV, with in-depth analysis of the individual peaks for studying chemical effects taking longer.

In addition to testing the surface chemistry of the bulk materials, samples are treated to remove possible chemical leachables and residues for additional testing using Fourier transform infrared spectroscopy (FTIR). FTIR is an analytical technique that measures the adsorption of infrared radiation by the sample versus the applied wavelength. When a sample is irradiated with infrared radiation, the adsorbed IR radiation excites molecules into higher vibrational states. The wavelength of light adsorbed by a particular molecule is a function of the energy difference between at-rest and excited vibrational states, and is characteristic of its molecular structure. The infrared adsorption bands can therefore be used to identify the molecular components. Materials samples are incubated in de-ionized water at 37° C. for 72 hrs, and any chemical residues and/or material leachables are identified using FTIR.

Materials are re-sterilized five times, and the chemical characterization of the materials is determined by the techniques described above to assess the impact of repeated sterilization on the chemical composition of the material, particularly with regards to the potential for accumulating chemical residuals.

### Experiment 4

### Biocompatability Tests

Chlorine dioxide sterilized materials are evaluated for cytotoxic chemical residuals, and modified material biocompatibility using established in vitro methods. Phase II biocompatibility tests include the remaining tests that are required for the FDA (based upon ISO Standards).

In vitro Extract Cytotoxicity: Materials are sterilized according to the protocol developed in Specific Aim #1. To test for the biocompatibility of residues and/or leachables from the sterilized materials, the chlorine dioxide sterilized material are incubated in culture medium, at 37° C. in a humidified 5% CO₂ atmosphere for 72 hours, under sterile conditions. The eluate is stored at 4° C. until used. The eluate for negative controls is prepared from high-density polyethylene. Positive controls are using dilutions of phenol. Human fibroblasts are seeded at 10⁵ cells/cm² in 24 well plates, and cultured at 37° C. in a humidified 5% CO₂ atmosphere for 24 hours. The culture medium is removed, and replaced with the eluate, and dilutions of the eluate. The fibroblasts continue to be cultured at 37° C. in a humidified 5% CO₂ atmosphere for a further 24, 48 and 72 hours. The dilutions of eluate:cell media are 1:1, 1:2, 1:4, and 1:8. At 24, 48, and 72 hours, the morphology of the cultures is assessed using the phase contrast light microscope, with assessment of general cell morphology, vacuolization, detachment, cell lysis, and membrane integrity.
In vitro Material Cytotoxicity: Human fibroblasts are seeded at 10⁵ cells/cm² in 24 well plates, and are cultured at 37° C. in a humidified 5% CO₂ atmosphere for 24 hours. After 24 hours, chlorine dioxide sterilized materials and control materials are placed on the cell monolayer, and incubated at 37° C. in a humidified 5% CO₂, atmosphere for 24, 48 and 72 hours. For negative controls, sterile high-density polyethylene are used, while the positive control material are organo-tin stabilized poly(vinylchloride), an ISO standard material is used for direct contact Cytotoxicity studies. At 24, 48, and 72 hours, the morphology of the cultures are assessed using the phase contrast light microscope, with assessment of general cell morphology, vacuolization, detachment, cell lysis, and membrane integrity. These studies utilize the three concentration/time variable pairs and evaluate effects on material structure.

### Evaluate the Efficacy of a Prototype Commercial Sterilization Unit and Disposable Gas Generation System Using Reusable Medical Instruments as a Test System.

Respirator and short procedure surgical packs were used in this series of experiments. Studies evaluated the sterilization capabilities of the prototype unit using reusable medical instruments as test articles. The instruments were prepared for sterilization using standard techniques and sporicidal effectiveness determined.

### Experiment 5

### Efficacy of a Prototype Sterilizer Using Reusable Medical Instruments

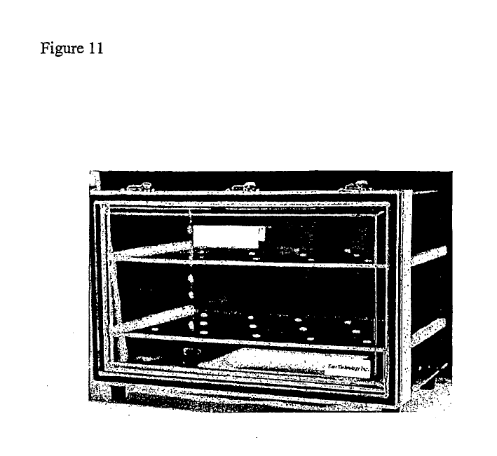

The prototype sterilization cabinet illustrated in FIG. 11 was evaluated using two test systems. The first was an oxygen respirator mask commonly used in long term care facilities. The second test article was a short procedure surgical pack prepared for sterilization using conventional means. The test articles were evaluated in two separate series of ClO₂ exposures. The respirator was placed in the sterilization cabinet with three B. subtilus spore strips attached to three different surfaces to include the bottom, top and inner surface of the respirator. Exposure to ClO₂ included the three time/concentration variable pairs identified above. Sporicidal activity was assessed as previously described.

The surgical instrument pack, consisting of a stainless steel tray and instruments, was prepared for sterilization with the inclusion of five spore strips within the inner tray. The tray and instruments were wrapped in two layers of sterilization cloth. The pack was then placed in the sterilization unit and subjected, in successive studies to the three time/concentration variables. Sporicidal activity was subsequently assessed as previously described.

Tamper proof interlocking mechanisms to avoid premature opening of sterilization cabinets during processing were necessary.

Although, compared to ethylene oxide, chlorine dioxide is of lesser toxicity the need to design and build a gas scrubbing system into the sterilizer is also envisioned.

Light Activated Chemistry

Chlorine dioxide was produced when a light source was applied to a hydrogel matrix which contains sodium chlorite and a photoacid generating (PAG) chemical. The acid generated by the PAG reacted with the sodium chlorite to produce chlorine dioxide gas. The hydrogel controls the diffusion rate of the chlorine dioxide out of the hydrogel as it is generated, forming a sustained antimicrobial environment. Also, the hydrogel acts as a scaffold to contain the chlorine dioxide reagents and attach them to the surfaces of established medical devices.

Sporicidal Activity of Chlorine Dioxide

The sporicidal activity of chlorine dioxide was demonstrated using washers inoculated with spores and exposed to different concentrations of a gas phase of chlorine dioxide. Sterile washers were inoculated with 1.4×10⁷ Bacillus subtilis var. niger spores and allowed to dry overnight. Solutions were prepared in sterile jars to obtain different chlorine dioxide levels. Five washers were suspended in each jar and exposed to one chlorine dioxide concentration for 4 hours in the dark. Two washers from each chlorine dioxide level were enumerated for log reduction and three washers from each chlorine dioxide concentration were analyzed for sterility. Log reduction samples were analyzed by placing one washer into 10 ml sterile Difco DE Neutralizing Broth, shaking for approximately 30 seconds and spread plating, in duplicate, onto Difco Trypticase Soy agar plates incubated at 35° C. for 48 hours. Sterility was determined by placing one washer in 50 ml sterile Difco Trypticase Soy Broth incubated at 35° C. for 14 days and visually checked daily for turbidity. Appropriate positive and negative controls were run with test samples. The results of the study show that Bacillus subtilis var. niger was recovered from the gas phase chlorine dioxide levels 50 ppm and 100 ppm. The 100 ppm concentration however, showed greater than a 6 log reduction in the Bacillus organism and the washers analyzed for sterility at this level showed no turbidity. No Bacillus subtilis var. niger was recovered after exposure to the various chlorine dioxide levels tested above 100 ppm. The results are summarized in Table 7.

TABLE 7
Sporicidal effects of gas phase C1O₂

| | | CFU/ml | | |
|---|---|---|---|---|
| | | Recovered | | |
| | | Bacillus | | |
| | | subtilis | Log | Sterility* |
| Description | Sample #^{a} | var. niger | Reduction^{b} | (Day 14) |
| Positive Control | 0A | NT | NA | No |
| Autoclaved washer, Inoculated | 0B | NT | NA | No |
| Dried overnight, No Exposure | 0C | NT | NA | No |
| | 0D | 11,000,000 | NA | NT |
| | 0E | 17,000,000 | NA | NT |
| Negative Control | 1A | NT | NA | Yes |
| Autoclaved washer, Uninoculated | 1B | NT | NA | Yes |
| No Exposure | 1C | NT | NA | Yes |
| | 1D | <1 | NA | NT |
| | 1E | <1 | NA | NT |
| Positive Control | 2A | NT | NA | No |
| Autoclaved washer, Inoculated | 2B | NT | NA | No |
| Dried overnight | 2C | NT | NA | No |
| Exposure to System with No Chlorine | 2D | 6,800,000 | NA | NT |
| Dioxide | 2E | 14,000,000 | NA | NT |
| 50 ppm Chlorine Dioxide | 3A | NT | NA | No |
| Autoclaved washer, Inoculated | 3B | NT | NA | No |
| Dried overnight | 3C | NT | NA | No |
| Exposure to 50 ppm | 3D | 50,000 | 2.30 | NT |
| Chlorine Dioxide for 4 hours | 3E | 73,000 | 2.13 | NT |
| 100 ppm Chlorine Dioxide | 4A | NT | NA | Yes |
| Autoclaved washer, Inoculated | 4B | NT | NA | Yes |
| Dried overnight | 4C | NT | NA | Yes |
| Exposure to 100 ppm | 4D | <1 | >6.99 | NT |
| Chlorine Dioxide for 4 hours | 4E | 5 | 6.29 | NT |
| 200 ppm Chlorine Dioxide | 5A | NT | NA | Yes |
| Autoclaved washer, Inoculated | 5B | NT | NA | Yes |
| Dried overnight | 5C | NT | NA | Yes |
| Exposure to 200 ppm | 5D | <1 | >6.99 | NT |
| Chlorine Dioxide for 4 hours | 5E | <1 | >6.99 | NT |
| 300 ppm Chlorine Dioxide | 6A | NT | NA | Yes |
| Autoclaved washer, Inoculated | 6B | NT | NA | Yes |
| Dried overnight | 6C | NT | NA | Yes |
| Exposure to 300 ppm | 6D | <1 | >6.99 | NT |
| Chlorine Dioxide for 4 hours | 6E | <1 | >6.99 | NT |
| 400 ppm Chlorine Dioxide | 7A | NT | NA | Yes |
| Autoclaved washer, Inoculated | 7B | NT | NA | Yes |
| Dried overnight | 7C | NT | NA | Yes |
| Exposure to 400 ppm | 7D | <1 | >6.99 | NT |
| Chlorine Dioxide for 4 hours | 7E | <1 | >6.99 | NT |

*Yes indicates no turbidity was observed, sample is sterile. No indicates turbidity observed, sample is not sterile.
NT = Not Tested
NA = Not Applicable
^{a}A, B, C indicate washers analyzed for sterility. D, E indicate washers analyzed for log reduction.
^{b}Log average was calculated using data for 2D and 2E

Chlorine Dioxide Production Using a Photoacid Generating (PAG) Chemistry

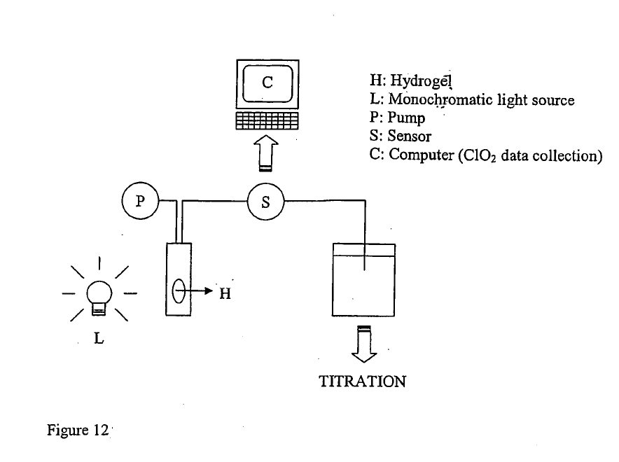

Preliminary studies were conducted to assess the ability of a selection of PAG chemistries to generate chlorine dioxide. The PAG chemistry produced an acid when activated by light sources with wavelengths in the order of 300-400 nm. The purpose of these studies was to determine if the acid generated by these dyes could be used to generate chlorine dioxide from NaClO₂, and what conditions were required for ClO₂ to be produced. The experimental setup is represented in FIG. 12.

The different PAG chemistries and NaClO₂ were weighed and ground together, before being placed in a glass scintillation vial. Small amounts of milli-Q H₂O and ethanol were added and the vial covered with a rubber septum. The glass vial was connected to a peristaltic pump and chlorine dioxide sensor, which were turned on prior to exposing the system to sunlight. Control experiments with only the NaClO₂ and milli-Q H₂O and ethanol were conducted. An experimental summary and the levels of chlorine dioxide generated are presented in Table 8.

TABLE 8
Experimental Summary

| | Amount | Amount | | | Max. |
|---|---|---|---|---|---|
| | PAG | NaClO₂ | Additional | Method of | ClO₂ |
| PAG | (mg) | (mg) | Reagents | Activation | (ppm) |
| Bis(styryl)benzene | 5.31 | 10.32 | Milli-Q H₂O, | Sunlight | 16 |
| | | | ethanol | | |
| Diphenyliodonium | 5.35 | 11.42 | Milli-Q H₂O, | Sunlight | 5.1 |
| dimethoxyanthracenesulfonate | | | ethanol | | |
| Triphenylamine | 5.81 | 10.42 | Milli-Q H₂O, | Sunlight | >250 |
| dimethylsulfate | | | ethanol | | |
| — | — | 10.32 | Milli-Q H₂O, | Sunlight | 3.1 |
| | | | ethanol | | |

### Background:

Two hydrogels were utilized as the matrix for the chlorine dioxide reagents. The formulations for each of the gels have been established in the literature for controlled drug delivery, and will be modified only if the results from these experiments dictate. Poly(vinyl alcohol) (PVA) hydrogels have been utilized for many biomedical applications, including controlled drug delivery (see Hassan, C. M., Stewart J. E., Peppas N. A., “Diffusional Characteristics of Freeze Thawed poly(vinyl alcohol) Hydrogels: Applications to Protein Controlled Release from Multiaminate Devices: European Journal of Pharmaceutics and Biopharmaceutics 49: 161-165, 2000). PVA hydrogels are formed using a repeated freeze-thawing technique. Poly(ethylene glycol) (PEG) has been established as a biocompatible polymer that has been utilized as a surface coating for medical implants to improve blood compatibility. PEG-based hydrogels are used in wound care products, cell encapsulation, and in the design of new drug delivery systems (see Zimmermann, J., Bittner, K., Stark, B., Mulhaupt, R., “Novel Hydrogels as Supports for in vitro Cell Growth: poly(ethylene glycol)- and Gelatin-based (meth)acrylamidopeptide Macromonomers”, Biomaterials 23: 2127-2134, 2002). The PEG hydrogel that was used was copolymerized with gelatin. The generation of chlorine dioxide was assessed using a monochromatic light source with a wavelength between 300-400 nm, which is known to activate the PAG chemistry and produce acid.

### Experiment 6

### Construction of Hydrogel

For each of the hydrogels, different amounts of sodium chlorite and the photo acid generating (PAG) chemistry were incorporated, to isolate a hydrogel formulation. To each of the pre-polymerized hydrogels, the amounts of sodium chlorite and PAG dye specified in Table 8 were added. The pre-polymerized hydrogels with the chlorine dioxide generating reagents were dip coated onto 6 mm discs of polyethylene terephthalate) (PET) and polymerized. 15% (w/v) PVA hydrogels will be made in deionized water and polymerized with 4 freeze-thaw cycles (freezing 8 hrs at −18° C., thawing for 4 hrs at −4° C.). PEG-gelatin solutions consisted of 10% gelatin, 6% NPC-PEG and 10% sucrose at pH 4.0. The solution was heated at 45° C. for 15 min to dissolve gelatin, and incubated at 4° C. for 15 min. The PEG hydrogels were polymerized by immersion in 200 mM Borate buffer (pH 8.5) for 1 hr. Residual p-nitrophenol was removed from the gels by continual washing in 10% sucrose solutions (pH 4.0) until the absorbance of the solutions at 400 nm is negligible. A summary of the hydrogel compositions and a selection of ratios of NaClO₂/PAG chemistry are outlined in Table 9. Each of the hydrogel formulations will be evaluated in triplicate.

TABLE 9
Composition of hydrogels

| | | Sodium Chlorite/Photo |
|---|---|---|
| | | Acid Generating |
| Hydrogel | Gel Concentrations | Chemistry (mg) |
| PVA | 15% (in deionized water) with 4 | 5/5, 10/5, 15/10 |
| | freeze- thaw cycles | |
| PEG | Ratio of PEG-gelatin hydrogels (%): | 5/5, 10/5, 15/10 |
| | gelatin:PEG:sucrose: 10:6:10 | |

### Experiment 7

Evaluation of the chlorine dioxide generating hydrogels of the hydrogels developed in Experiment 6 were evaluated to determine the amount of chlorine dioxide generated and released from the hydrogel. The experimental set up is represented in FIG. 12. Individual samples were placed in an optical glass cuvette, and exposed to a monochromatic light source of 300-400 nm to activate the PAG chemistry. The protons released by the PAG chemistry oxidize the NaClO₂ and produce ClO₂ gas. As the gas diffuses out of the hydrogel, it was pumped out of the cuvette and through a chlorine dioxide sensor, and into a potassium iodide+acid solution, analyzed for total chlorine dioxide concentrations.

The oxidation of iodide to iodine by chlorine dioxide gas is represented by Equation 2, while the reduction of iodine back to iodide by the sodium thiosulfate titrant is given by Equation 3.

ClO₂+5I⁻+4H⁺→2.5I₂+Cl⁻+2H₂O [Eqn. 2]

2Na₂S₂O₃+I₂→2I⁻+Na₂S₄O₆+2Na⁺ [Eqn. 3]

Equation 4 is used calculate the total chlorine dioxide produced:

V(ClO₂)=Vtitrant×Ctitrant×22.4×1000/5 [Eqn. 4]

V(ClO₂)=volume ClO₂ produced (μl)

Vtitrant=volume of sodium thiosulfate titrant

Ctitrant=concentration of normalized sodium thiosulfate titrant

### Experiment 8

Each of the hydrogels developed in Experiment 6 were evaluated to determine the amount of chlorine dioxide generated and released from the hydrogel when the activating light source used is a broad-band light source. The experimental set up is represented in FIG. 12, and follows the same experimental procedure as Experiment 8. Individual samples were placed in an optical glass cuvette, and exposed to a selection of different light sources activate the formation of chlorine dioxide gas. The gases were pumped out of the cuvette and through a chlorine dioxide sensor, and into a potassium iodide+acid solution, which was analyzed for total chlorine dioxide concentrations. The light sources that included varying intensities of fluorescent light. For each of the broad-band light sources, the spectral characterization was determined. The same calculations specified in Experiment 7 (Equation 3) were used to calculate the total amount of ClO2 produced.

Background: To assess the antimicrobial activity of the chlorine dioxide being generated by the hydrogels, the ability of the hydrogels to generate a zone of inhibition (ZOI) on an agar plate seeded with bacteria was assessed. ZOI measurements are a standard method used to assess the antimicrobial activity of agents. The hydrogels were tested against a selection of bacteria, selected on the basis of their prevalence as pathogenic agents associated with medical implants. The chlorine dioxide generating hydrogels were compared to control samples, including negative controls of a hydrogel with no chlorine dioxide generating agents incorporated, chlorine dioxide generating hydrogels that have been exhausted of chlorine dioxide, and hydrogels that contain only NaClO₂ (no PAG). Positive controls included antibiotic discs.

### Experiment 9

Zone of Inhibition Study: Overnight cultures of the bacteria listed in Table 10 were grown inappropriate growth media and incubated at 37° C. overnight 10 μl of the overnight cultures were plated onto individual agar plates to create isolated colony forming units (CFUs). The plates were incubated at 37° C. overnight. From the overnight culture plates, a single CFU were plated across a Mueller-Hinton agar plate. One material sample will be placed on each plate. The plates were incubated at 37° C. for 24 hours with an activating light source applied to the material for the duration of the incubation period. Images of the plates were captured after a 24 hour incubation period, and the ZOI measured for each sample. Each sample was tested in triplicate.

TABLE 10
Bacteria being tested against the hydrogel
Bacteria strains

Staphylococcus epidermidis
Staphylococcus aureus (MR Strain)
Escherichia coli
Pseudomonas aeruginosa

The entire contents of each of U.S. provisional applications 60/560,909 and 60/561,698 filed on Apr. 13, 2004 and Apr. 9, 2004, respectively are incorporated herein by reference.

Obviously, numerous modifications and variations of the present invention are possible in light of the above teachings. It is therefore to be understood that within the scope of the appended claims, the invention may be practiced otherwise than as specifically described herein.

1. A portable gas sterilization unit, comprising:
a) a chamber comprising (i) a shell and (ii) at least one door,
wherein the shell has an oxidizer-resistant inner surface;
wherein the shell and door are made from a solid rigid material and wherein the shell and door may be closed to enclose a gas-tight environment in the chamber;
wherein the door is connected to the shell and seals the chamber to maintain a gas tight seal capable of maintaining a pressure of up to 2 psi for at least 24 hours,
wherein the door has a lock engageable with the door and controlled by a locking mechanism;
wherein the locking mechanism may or may not be interfaced with an electronic measuring system to prohibit opening of the door when chlorine dioxide gas is present in the chamber;
b) a chlorine dioxide detector connected to the enclosed environment inside the chamber for measuring the concentration of the chlorine dioxide between 0 and 3,000 ppm inside the chamber;
c) a disposable chlorine dioxide generator for generating chlorine dioxide and connected to the chamber through a flow pump for circulating the chlorine dioxide from the chlorine dioxide generator to the chamber; and
d) a chemical quencher attached to the chamber by a tube.

2. The portable gas sterilization unit of claim 1, wherein the shell is plastic, stainless steel, aluminum or a combination of these materials.

3. The portable gas sterilization unit of claim 1, wherein the locking mechanism is electronically connected to a chlorine dioxide sensor; and
the locking mechanism maintains the door in a closed, locked and gas-sealed position while the concentration of chlorine dioxide is above a threshold level.

4. The portable gas sterilization unit of claim 1, wherein the chlorine dioxide sensor is either an electrochemical sensor or an optical sensor

5. The portable gas sterilization unit of claim 1, wherein the chlorine dioxide gas generator is an external system connected to the interior of the chamber, and
the gas generator contains one or more liquid or dry chemical reagents.

6. The portable gas sterilization unit of claim 1, further comprising an air circulation system for recirculating the chlorine dioxide gas through the chamber.

7. The portable gas sterilization unit of claim 1, further comprising a gas absorbing unit for removing the chlorine dioxide gas from the environment in the chamber.

8. The portable gas sterilization unit of claim 1, wherein a substance that reduces susceptibility to oxidation and degradation by chlorine dioxide is present on the oxidizer-resistant surface of the shell.

9. The portable gas sterilization unit of claim 2, wherein the oxidizer-resistant surface is spray-coated or electroplated onto the solid rigid material.

10. The portable gas sterilization unit of claim 2, wherein the oxidizer-resistant surface is a fluorocarbon material.

11. The portable gas sterilization unit of claim 3, wherein the locking mechanism is a mechanical interlocking system.

12. The portable gas sterilization unit of claim 4, wherein the chorine dioxide detector is an electrochemical cell comprising at least one of gold or platinum electrodes.

13. The portable gas sterilization unit of claim 4, wherein the chorine dioxide detector is an optical cell capable of evaluating absorption changes of gas in the chamber calibrated to provide a measurement of chlorine dioxide concentration in the chamber.

14. The portable gas sterilization unit of claim 5, wherein the disposable chlorine dioxide gas generator contains one or more chlorine-containing compounds selected from the group consisting of sodium chlorite and sodium chlorate.

15. The portable gas sterilization unit of claim 5, wherein the disposable chlorine dioxide gas generator contains an acidic compound.

16. The portable gas sterilization unit of claim 5, wherein the disposable chlorine dioxide gas generator contains a photochemical compound that releases an acidic compound upon exposure to light.

17. The portable gas sterilization unit of claim 5, wherein the reagents are aqueous solutions.

18. The portable gas sterilization unit of claim 14, wherein the chlorine-containing compounds are aqueous solutions.

19. The portable gas sterilization unit of claim 15, wherein the acidic compound is at least one selected from the group consisting of hydrochloric acid, citric acid, ascorbic acid, tartaric acid and boric acid.

20. The portable gas sterilization unit of claim 15, wherein the acidic compound is a photo acid generating compound susceptible to absorptions of multiple photons and undergoes reaction with high efficiency to form one or more Lewis acidic species.

21. The portable gas sterilization unit of claim 20, wherein the photo acid generating compound is bonded to or within a polymer.

22. The portable gas sterilization unit of claim 21, wherein the polymer is polyethylene, polycarbonate, polyethylene or polyvinylchloride.

23. The portable gas sterilization unit of claim 20, wherein the acid generated by the photo acid reacts with either sodium chlorite, sodium chlorate or both chlorite and sodium chlorate, to form chlorine dioxide.

24. A two photon photo-activated chlorine dioxide generator system, comprising:
a chlorine component comprising at least one of sodium chlorite and sodium chlorate; and
a photo acid component comprising at least one two photon photo acid generating compound,
wherein the chlorine component and the photo acid component are intermixed in a mixture, and
wherein the mixture releases chlorine dioxide gas after exposure to light.

25. The two photon photo-activated chlorine dioxide generator system of claim 24, wherein the photo acid component comprises diphenyliodonium 9,10-dimethoxyanthracenesulfonate.

26. The two photon photo-activated chlorine dioxide generator system of claim 24, wherein the chlorine component and the photo acid component are dispersed within a solid matrix.

27. The two photon photo-activated chlorine dioxide generator system of claim 24, wherein the solid matrix is a polymer.

28. The two photon photo-activated chlorine dioxide generator system of claim 24, wherein the chlorine component and the photo acid component are present as a homogeneous liquid mixture.

29. A method for making chlorine dioxide, comprising:
mixing at least one of sodium chlorite and sodium chlorate with at least one two photon photo acid generating compound to form a mixture, and
exposing the mixture to light.

30. The method of claim 29, wherein the photo acid component comprises diphenyliodonium 9,10-dimethoxyanthracenesulfonate.

31. A chlorine dioxide generating kit, comprising:
a first composition comprising at least one of sodium chlorite and sodium chlorate; and
a second composition comprising at least one two photon photo acid generating compound,
wherein the first composition is present in a first package and the second composition is present in a second package and the first and second packages separate the first and second compositions,
wherein the first package is inside the second package or the second package is within the first package,
wherein the inside package may be broken to allow the first and second composition to mix, and
wherein the mixture of the first and second compositions forms chlorine dioxide after exposure to light.
